(19) United States
(12) Patent Application Publication
(10) Pub. No.: US 2011/0160659 A1
(43) Pub. Date: Jun. 30, 2011

(54) Drug-Delivery Balloons

(75) Inventors: John T. Clarke, Co. Galway (IE); Jan Weber, Maastricht (NL); Aiden Flanagan, Co. Galway (IE)

(73) Assignee: BOSTON SCIENTIFIC SCIMED, INC., Maple Grove, MN (US)

(21) Appl. No.: 12/961,927

(22) Filed: Dec. 7, 2010

Related U.S. Application Data
(60) Provisional application No. 61/291,048, filed on Dec. 30, 2009.
(60) Provisional application No. 61/292,924, filed on Jan. 7, 2010.

(51) Int. Cl. A61M 25/10; A61L 29/16

(52) U.S. Cl. 604/103.02

(57) ABSTRACT

Drug-delivery balloons, as well as related medical devices and methods, are disclosed.

### CROSS-REFERENCE TO RELATED APPLICATION

This application claims priority under 35 USC §119(e) to U.S. Provisional Patent Application Ser. No. 61/291,048, filed on Dec. 30, 2009, and to U.S. Provisional Patent Application Ser. No. 61/292,924, filed on Jan. 7, 2010, the entire contents of both are hereby incorporated by reference.

### TECHNICAL FIELD

This disclosure relates to drug-delivery balloons, as well as related medical devices and methods.

### BACKGROUND

The body includes various passageways such as blood vessels (e.g., arteries) and body lumens. These passageways sometimes become occluded (e.g., by a tumor or plaque). To widen an occluded body vessel, balloon catheters can be used, e.g., in angioplasty.

A balloon catheter can include an inflatable and deflatable balloon carried by a long and narrow catheter body. The balloon can be initially folded around the catheter body to reduce the radial profile of the balloon catheter for easy insertion into the body.

During use, the folded balloon can be delivered to a target location in the vessel, e.g., a portion occluded by plaque, by threading the balloon catheter over a guide wire emplaced in the vessel. The balloon is then inflated, e.g., by introducing a fluid (such as a gas or a liquid) into the interior of the balloon. Inflating the balloon can radially expand the vessel so that the vessel can permit an increased rate of blood flow. After use, the balloon is typically deflated and withdrawn from the body.

### SUMMARY

Generally, this disclosure relates to a medical device (e.g., a balloon catheter) that includes a drug-delivery balloon containing a layer of a therapeutic agent (e.g., a drug) on the outer surface of the balloon wall. The layer can include a bioadhesive polymer that is capable of adhering or binding to a tissue at a target site (e.g., a wall) in a blood vessel (e.g., a coronary blood vessel) so that it can hold the therapeutic agent at the target site for an extended period time (e.g., for at least about 14 days) to allow sufficient uptake of the therapeutic agent by the tissue at the target site.

In one aspect, this disclosure features a medical device that includes a drug-delivery balloon. The drug-delivery balloon includes a balloon wall having an outer surface and a first layer supported by the outer surface. The first layer contains a plurality of particles, in which each particle includes a therapeutic agent and a polymeric carrier. The polymeric carrier contains a polymer capable of adhering or binding to a tissue on a wall of a blood vessel.

In another aspect, this disclosure features a medical device that includes a drug-delivery balloon. The drug-delivery balloon includes a balloon wall having an outer surface and a first layer supported by the outer surface. The first layer includes a plurality of fibers, in which each fiber includes a therapeutic agent and a polymeric carrier.

In another aspect, this disclosure features a medical device that includes a drug-delivery balloon. The drug-delivery balloon includes a balloon wall having an outer surface and a first layer supported by the outer surface. The first layer includes a therapeutic agent and a polymeric carrier. The polymeric carrier contains a polymer capable of adhering or binding to a tissue on a wall of a blood vessel.

In still another aspect, this disclosure features a medical device that includes a drug-delivery balloon. The drug-delivery balloon includes a balloon wall having an outer surface and a first layer supported by the outer surface. The first layer includes a therapeutic agent and a polymeric carrier. The polymer carrier is capable of maintaining at least about 25 wt % of the therapeutic agent at a target site in a blood vessel for at least about 14 days after the balloon is withdrawn from the blood vessel.

Embodiments of the above-mentioned medical devices can have one or more of the following features.

Each particle or fiber can include a core encapsulated by a shell, in which the core can include the therapeutic agent and the shell can include the polymeric carrier. Alternatively, the therapeutic agent can be dispersed in the polymeric carrier in each particle or fiber.

The plurality of particles or fibers can have an average diameter of from about 20 nm to about 1,000 nm (e.g., from about 20 nm to about 150 nm, from about 100 nm to about 150 nm, or from about 150 nm to about 1,000 nm).

The polymer can be a biodegradable polymer.

The polymeric carrier can include a polyhydroxyalkanoate, a polylactone, a polylactic acid, polyglycolic acid, a cyanoacrylate-based polymer, a polyacrylate, a poly(vinyl alcohol), a poly(ethylene glycol), or a copolymer or mixture thereof. For example, the polymeric carrier can include a polyhydroxybutyrate, a polylactic acid, a poly(methyl methacrylate), a poly(vinyl alcohol), a poly(ethylene glycol), or a copolymer or mixture thereof.

The polymer can have a number average molecular weight of from about 10,000 g/mol to about 75,000 g/mol.

The polymer can have a shear viscosity of from about 5,000 centipoises to about 2×10⁶ centipoises.

The first layer can have an elastic modulus of from about 10 kPa to about 10 MPa.

The polymer carrier can be capable of maintaining at least about 25 wt % of the therapeutic agent at a target site in a blood vessel for at least about 14 days after the balloon is withdrawn from the blood vessel.

The therapeutic agent can be therapeutically effective in inhibiting restenosis. For example, the therapeutic agent can include paclitaxel, everolimus, or a derivative thereof.

The therapeutic agent can be in a crystalline form.

Each particle or fiber can include from about 5 wt % to about 90 wt % of the therapeutic agent.

The first layer can be a discontinuous layer.

The balloon can further include a second layer between the first layer and the outer surface of the balloon wall, where the second layer is capable of inhibiting binding of the first layer to the outer surface of the balloon wall. For example, the second layer can include a poly(ethylene glycol), a phospholipid, or a metal.

At least about 50 wt % of the therapeutic agent can remain on the outer surface of the balloon wall when the balloon reaches a target site in a blood vessel.

The balloon can be capable of transferring at least about 20 wt % of the therapeutic agent to a target site in a blood vessel.

Embodiments and/or aspects can provide one or more of the following advantages.

The bioadhesive polymer can significantly reduce the amount of the therapeutic agent washed off the outer surface of the balloon wall by the blood flow and significantly increase the amount of the therapeutic agent on the outer surface of the balloon when the balloon reaches a target site in a blood vessel (e.g., a tissue on a blood vessel wall). The bioadhesive polymer can also significantly increase the amount of the therapeutic agent transferred from the balloon to a target site in a blood vessel. The bioadhesive polymer can significantly increase the time the therapeutic agent remains at a target site in blood vessel after delivery. Finally, the amount of the therapeutic agent required to be coated on the balloon can be significantly reduced while still providing the desired therapeutic effect.

All publications, patent applications, patents, and other references mentioned herein are incorporated by reference herein in their entirety.

The details of one or more embodiments of the disclosure are set forth in the accompanying drawings and the description below. Other features and advantages of the invention will be apparent from the description, drawings, and claims.

### DESCRIPTION OF DRAWINGS

FIG. 1 is a cross-sectional view of a portion of an exemplary balloon having a layer of a plurality of particles disposed on its wall, in which each particle contains a therapeutic agent and a polymeric carrier.

FIG. 2 is a cross-sectional view of an exemplary balloon in a folded state within an occluded vessel.

FIG. 2A is an end view of the balloon shown in FIG. 2 in the vessel.

FIGS. 3 and 3A illustrate the balloon shown in FIGS. 2 and 2A in an expanded state.

FIGS. 4 and 4A illustrate the balloon shown FIGS. 2 and 2A in a refolded state.

Like reference symbols in the various drawings indicate like elements.

### DETAILED DESCRIPTION

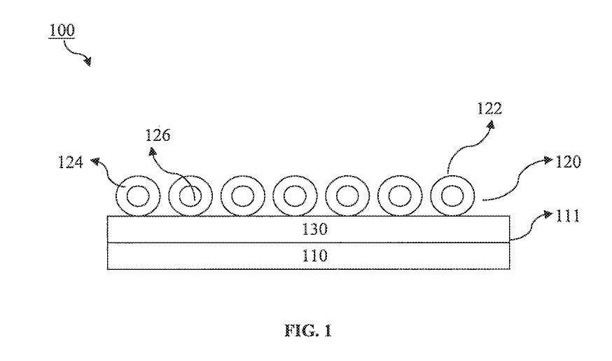

FIG. 1 shows a cross-sectional view of a portion of an exemplary balloon 100 having a balloon wall 110 with an outer surface 111, a first layer 120 on outer surface 111, and optionally a second layer 130 between balloon wall 110 and first layer 120. First layer 120 includes a plurality of particles 122, each of which contains a polymeric carrier 124 and a therapeutic agent 126.

In the embodiments shown in FIG. 1, each particle 122 includes a core encapsulated by a shell, in which the core includes therapeutic agent 126 and the shell includes polymeric carrier 124. In certain embodiments, each particle 122 can include therapeutic agent 126 dispersed (e.g., uniformly dispersed) in polymeric carrier 124. In such embodiments, each particle 122 can have a core-shell structure in which an inert core (e.g., an inert core made of sugar particles) is surrounded by a shell containing therapeutic agent 126 dispersed in polymeric carrier 124. Alternatively, therapeutic agent 126 can be dispersed in polymeric carrier 124 without forming a core-shell structure.

Polymeric carrier 124 typically includes a bioadhesive polymer, i.e., a polymer capable of adhering or binding to a tissue on a wall of a blood vessel. For example, a bioadhesive polymer can bind to a protein in a tissue on a blood vessel wall upon contacting the vessel wall. The bioadhesive polymer can adhere or bind to a blood vessel tissue through any suitable means, such as chemical bonding (e.g., covalent bonding, hydrogen bonding, or ionic bonding) or mechanical adhesion. Examples of such bioadhesive polymers include polyhydroxyalkanoates, polylactones (e.g., polycaprolactones), polylactic acids (e.g., poly(D-lactic acid)s or poly(L-lactic acid)s), polyglycolic acids (e.g., poly(lactic-co-glycolic acid)s), cyanoacrylate-based polymers (e.g., poly(methyl 2-cyanoacrylate)s or poly(ethyl 2-cyanoacrylate)s), polyacrylates (e.g., poly(methyl methacryalte)s), poly(vinyl alcohol)s, poly(ethylene glycol)s, or copolymers or mixtures thereof. Exemplary polyhydroxyalkanoates include polyhydroxybutyrate, polyhydroxyvalerate, or poly(hydroxybutyrate-co-hydroxyvalerate). Other bioadhesive polymers have been described, for example, in (1) U.S. Pat. No. 6,221,316, (2) Biochemical et Biophysical Acta, 1123 (1992) 33-34, (3) Eur. Polymer J. 30 (1994) 1327-1333, and (4) Biomaterials, 26 (2005), pp. 661-670. In some embodiments, polymeric carrier 124 includes two or more (e.g., three, four, or five) of such bioadhesive polymers.

In general, the bioadhesive polymer is a biodegradable polymer. As used herein, the term “biodegradable polymer” refers to a polymer that can be broken down into harmless products inside body. The above-mentioned exemplary bioadhesive polymers are all biodegradable polymers.

The molecular weight and viscosity of the bioadhesive polymer can vary as desired. In general, the molecular weight of the bioadhesive polymer is sufficiently large to provide a suitable viscosity so as to maintain therapeutic agent 126 on the balloon surface and minimize the possibility of therapeutic agent 126 being washed off from the balloon surface by the blood flow in a blood vessel during delivery. In addition, the molecular weight of the bioadhesive polymer is sufficiently small so that the polymer can be easily dissolved or dispersed in a solvent to form a solution or dispersion. For example, the bioadhesive polymer mentioned herein can have a number average molecular weight of at least about 10,000 g/mol (e.g., at least 15,000 g/mol, at least 20,000 g/mol, at least 25,000 g/mol, or at least 30,000 g/mol) or at most about 75,000 g/mol (e.g., at most about 70,000 g/mol, at most about 65,000 g/mol, at most about 60,000 g/mol, or at most about 55,000 g/mol). The number average of molecular weight of the bioadhesive polymer can be measured by methods well known in the art, such as gel permeation chromatography. In some embodiments, the bioadhesive polymer mentioned herein can have a shear viscosity of at least about 5,000 centipoises (e.g., at least 10,000 centipoises, at least 50,000 centipoises, at least 100,000 centipoises, or at least 500,000 centipoises) or at most about 2×10⁶ centipoises (e.g., at most 1×10⁶ centipoises, at most 800,000 centipoises, at most 600,000 centipoises, or at most 400,000 centipoises). An example for measuring the shear viscosity has been described in Taherian et al., International Journal of Food Properties, Vol. 11, Issue 1, January 2008, pages 24-43.

In some embodiments, the bioadhesive polymer exhibits shear thinning properties. For example, the bioadhesive polymer can be pseudoplastic (i.e., the viscosity of the polymer decreases under shear stress) and thixotropic (i.e., the viscosity of the polymer decreases under shear stress and continues to decrease with time). For example, the shear viscosity of the bioadhesive polymer mentioned herein can be reduced to less than about 5,000 centipoises when shear stress is applied (e.g., when balloon wall 110 expands against a blood vessel wall). Shear thinning behavior has been described, for example, in Taherian et al., International Journal of Food Properties, Vol. 11, Issue 1, January 2008, pages 24-43.

Without wishing to be bound by theory, it is believed that the bioadhesive polymer can significantly reduce the amount of therapeutic agent 126 that is washed off from outer surface 111 of balloon wall 110 by the blood flow and significantly increase the amount of therapeutic agent 126 on outer surface 111 when balloon 100 reaches a target site in a blood vessel. In some embodiments, at least about 50 wt % (e.g., at least about 60 wt %, at least about 70 wt %, at least about 80 wt %, or at least about 90 wt %) of therapeutic agent 126 applied onto outer surface 111 before balloon 110 is inserted into a blood vessel remains on outer surface 111 when balloon 100 reaches a target site in a blood vessel. The amount of therapeutic agent 126 remaining on outer surface 111 can be determined by measuring the difference between the weight of balloon 100 before insertion into a blood vessel and the weight of balloon 100 after it reaches a target site in a blood vessel and is withdrawn from the blood vessel without being inflated.

Without wishing to be bound by theory, it is believed that the bioadhesive polymer can significantly increase the amount of therapeutic agent 126 transferred from balloon 100 to a target site in a blood vessel (e.g., a tissue on a blood vessel wall). In some embodiments, balloon 100 is capable of transferring at least about 20 wt % (e.g., at least about 30 wt %, at least about 40 wt %, at least about 50 wt %, or at least about 60 wt %) of the total amount of therapeutic agent 126 (i.e., the amount of therapeutic agent 126 applied onto outer surface 111 before balloon 110 is inserted into a blood vessel) to a target site in a blood vessel. The amount of therapeutic agent 126 transferred to a target site in a blood vessel can be determined in an animal experiment by measuring the amount of therapeutic agent 126 at the target site after balloon 100 is withdrawn from the blood vessel and the tested animal is sacrificed. An example of such a measurement method has been described in Balakrishnana et al., Journal of Controlled Release, Vol. 131, No. 3, Nov. 12, 2008, pages 173-180.

Without wishing to be bound by theory, it is believed that the bioadhesive polymer can significantly increase the time therapeutic agent 126 remains at a target site in blood vessel after delivery. In some embodiments, the bioadhesive polymer in polymeric carrier 124 is capable of maintaining at least about 25 wt % (e.g., at least about 30 wt %, at least about 35 wt %, at least about 40 wt %, at least about 45 wt %, at least about 50 wt %, at least about 60 wt %, or at least about 70 wt %) of therapeutic agent 126 at a target site in a blood vessel (e.g., by holding therapeutic agent 126 against the tissue at the target site) for at least about 14 days (e.g., at least about 30 days, at least about 60 days, at least 90 days, at least 180 days, or at least about 270 days) after the balloon is withdrawn from the blood vessel. The amount of therapeutic agent 126 remaining at a target site in a blood vessel can be determined in an animal experiment by delivering therapeutic agent 126 to a target site in a group of animals, sacrificing the animals at different time points, and measuring the amount of therapeutic agent 126 remaining at the target site. An example of such a measurement method has been described in Balakrishnana et al., Journal of Controlled Release, Vol. 131, No. 3, Nov. 12, 2008, pages 173-180.

In addition, as the bioadhesive polymer can reduce the amount of therapeutic agent 126 that is washed off by blood flow during a delivery process, increase the amount of therapeutic agent 126 transferred to a target site, and increase the time therapeutic agent 126 remains at the target site, the amount of therapeutic agent 126 required to be coated on balloon 110 can be significantly reduced while still provide the desired therapeutic effect.

In general, therapeutic agent 126 can be a genetic therapeutic agent, a non-genetic therapeutic agent, or cells. Therapeutic agent 126 can include a singular agent, or can include more than one (e.g., two, three, or four) agent. Therapeutic agent 126 can be nonionic, or can be ionic (e.g., anionic or cationic) in nature. Therapeutic agent 126 for a vascular application can be a drug that inhibits restenosis. A specific example of such a therapeutic agent is paclitaxel or derivatives thereof, e.g., docetaxel. Soluble paclitaxel derivatives can be made by tethering solubilizing moieties (e.g., COCH₂CH₂CONHCH₂CH₂(OCH₂)ₙOCH₃ (n being, e.g., 1 to 100 or more)) off the 2′ hydroxyl group of paclitaxel. Other water soluble derivatives of paclitaxel have been described in, for example, U.S. Pat. No. 6,730,699.

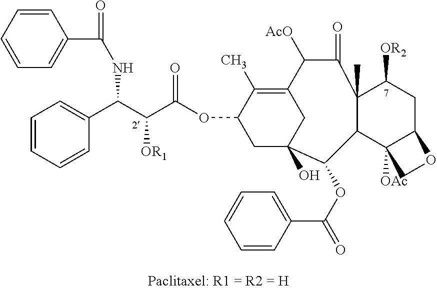

Exemplary non-genetic therapeutic agents include: (a) anti-thrombotic agents such as heparin, heparin derivatives, urokinase, PPack (dextrophenylalanine proline arginine chloromethylketone), and tyrosine; (b) anti-inflammatory agents, including non-steroidal anti-inflammatory agents (NSAID), such as dexamethasone, prednisolone, corticosterone, budesonide, estrogen, sulfasalazine and mesalamine; (c) anti-neoplastic/antiproliferative/anti-miotic agents such as paclitaxel, 5-fluorouracil, cisplatin, vinblastine, vincristine, epothilones, endostatin, angiostatin, angiopeptin, rapamycin (sirolimus), biolimus, tacrolimus, everolimus, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promoters; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation effectors; (n) vasodilating agents; (o) agents that interfere with endogenous vasoactive mechanisms; (p) inhibitors of leukocyte recruitment, such as monoclonal antibodies; (q) cytokines, (r) hormones; and (s) antispasmodic agents, such as alibendol, ambucetamide, aminopromazine, apoatropine, bevonium methyl sulfate, bietamiverine, butaverine, butropium bromide, n-butylscopolammonium bromide, caroverine, cimetropium bromide, cinnamedrine, clebopride, coniine hydrobromide, coniine hydrochloride, cyclonium iodide, difemerine, diisopromine, dioxaphetyl butyrate, diponium bromide, drofenine, emepronium bromide, ethaverine, feclemine, fenalamide, fenoverine, fenpiprane, fenpiverinium bromide, fentonium bromide, flavoxate, flopropione, gluconic acid, guaiactamine, hydramitrazine, hymecromone, leiopyrrole, mebeverine, moxaverine, nafiverine, octamylamine, octaverine, oxybutynin chloride, pentapiperide, phenamacide hydrochloride, phloroglucinol, pinaverium bromide, piperilate, pipoxolan hydrochloride, pramiverin, prifinium bromide, properidine, propivane, propyromazine, prozapine, racefemine, rociverine, spasmolytol, stilonium iodide, sultroponium, tiemonium iodide, tiquizium bromide, tiropramide, trepibutone, tricromyl, trifolium, trimebutine, tropenzile, trospium chloride, xenytropium bromide, ketorolac, and pharmaceutically acceptable salts thereof; or their derivatives.

Exemplary genetic therapeutic agents include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017 (SUPRATEK), lipids such as cationic lipids, liposomes, lipoplexes, nanoparticles, or micro particles, with and without targeting sequences such as the protein transduction domain (PTD).

In some embodiments, therapeutic agent 126 is in a crystalline form. Without wishing to be bound by theory, it is believed that therapeutic agent 126 in a crystalline form has a longer dissolution time in the aqueous medium in body and therefore exhibits a more extended release profile than that in an amorphous state.

The weight percentage of therapeutic agent 126 in each particle 122 can vary as desired. In some embodiments, each particle includes at least about 5 wt % (e.g., at least about 10 wt %, at least about 20 wt %, at least about 30 wt %, or at least about 40 wt %) or at most about 90 wt % (e.g., at most about 80 wt %, at most about 70 wt %, at most about 60 wt %, or at most about 50 wt %) of the therapeutic agent. When particles 122 include a small amount of therapeutic agent 126, therapeutic agent 126 can have an extended release time due to a large amount of polymeric carrier 124 in the particles. On the other hand, when particles 122 include a large amount of therapeutic agent 126, therapeutic agent 126 can be delivered to the target site in a large amount.

In some embodiments, particles 122 mentioned herein can have an average diameter of at least about 20 nm (e.g., at least about 100 nm, at least about 150 nm, or at least about 200 nm) or at most about 1,000 nm (e.g., at most about 900 nm, at most about 800 nm, or at most about 700 nm). The “particle diameter” and “particle diameter range” mentioned herein refers to those measured from particles taken up in a non-solvent (e.g., in a dispersion nor emulsion) by using a Malvern Mastersizer (i.e., a laser particle size analyzer). In some embodiments, particles 122 can have an average diameter of from about 20 nm to about 150 nm. Such a size is similar to those of viruses and lipoproteins, and therefore allows efficient uptake of therapeutic agent 126 in the particles by cells via endocytosis. In certain embodiments, particles 122 can have an average diameter of from about 100 nm to about 150 nm. Particles of such a size can be transfected into cells via clathrin-mediated endocytosis. In certain embodiments, particles 122 can have an average diameter of from about 150 nm to about 1,000 nm. Particles of such a size can be embedded in the intracellular space to facilitate uptake of therapeutic agent 126 by the cells.

In certain embodiments, particles 122 mentioned herein can have a narrow particle diameter range. For example, particles 122 can have a particle diameter range of 10-100 nm, 10-200 nm, 10-500 nm, 100-300 nm, 100-400 nm, 100-500 nm, 200-400 nm, 200-500 nm, 200-600 nm, 300-500 nm, 300-600 nm, 300-700 nm, 400-600 nm, 400-700 nm, 400-800 nm, 500-700 nm, 500-800 nm, 500-900 nm, 500-1,000 nm, 600-800 nm, 600-900 nm, 600-1,000 nm, 700-900 nm, 700-1,000 nm, or 800-1,000 nm. Without wishing to be bound by theory, it is believed that, by providing therapeutic agent 126 in a specific particle size range, the dosage at the target site in a blood vessel can be more predictable. In certain embodiments, therapeutic agent 126 including two or more sets of different narrow size range can be used to provide a desired bioavailability profile over time. For example, 50% of crystals in therapeutic agent 126 can have an average diameter of about 1,000 nm and the other 50% can have an average diameter of about 300 nm. Balloons coated with nanocrystal drugs of different particle sizes have been described, for example, in commonly-owned co-pending U.S. Provisional Application No. 61/224,723.

Particles 122 can form a single layer of particles or multiple layers of particles in first layer 120. Without wishing to be bound by theory, it is believed that first layer 120 having multiple layers of particles 122 can reduce the dissolution rate of first layer 120, thereby reducing the amount of therapeutic agent 126 being washed off from outer surface 111 before balloon 110 reaches a target site in a blood vessel.

First layer 120 can be either a continuous layer or a discontinuous layer. When first layer 120 is a discontinuous layer, it can be applied (e.g., sprayed) onto only certain discontinuous sections of balloon wall 110. An advantage of forming discontinuous first layer 120 on balloon wall 110 is that, if a portion of first layer 120 is washed off from balloon wall 110 during insertion of balloon 100 into a blood vessel, the loss can be limited to the section in which the portion of first layer 120 is located.

First layer 120 can have an elastic modulus depending on, for example, the nature and amount of the bioadhesive polymer used. For example, when measured by using an atomic force microscope, first layer 120 can have an elastic modulus at least about 10 kPa (e.g., at least about 50 kPa, at least about 100 kPa, at least about 500 kPa, or at least about 1 MPa) or at most about 10 MPa (e.g., at most about 8 MPa, at most about 6 MPa, at most about 4 MPa, or at most about 2 MPa). An example of measuring the elastic modulus of a layer using an atomic force microscope has been described in Moeller et al., “AFM Nanoindentation of Polymers” Microsc Microanal 13 (Suppl 2), 2007. In general, a rigid or a gel-like bioadhesive polymer forms a layer having a relatively low elastic modulus and a flexible bioadhesive polymer (e.g., a rubber) forms a layer having a relatively large elastic modulus.

First layer 120 can generally have any suitable thickness. For example, first layer 120 can have a thickness of at least about 0.1 μm (e.g., at least about 0.5 μm, at least about 1 μm, at least about 5 μm, or at least about 10 μm) or at most about 2,000 μm (e.g., at most about 1,500 μm, at most about 1,000 μm, at most about 500 μm, or at most about 100 μm). In general, first layer 120 can have a uniform thickness throughout the entire layer or can have different thicknesses at different portions of the layer.

Optionally, balloon 100 can have a second layer 130 between balloon wall 110 and first layer 120. In some embodiments, second layer 130 can include a material capable of inhibiting binding (e.g., non-specific binding) of first layer 120 to outer surface 111 of balloon wall 110, thereby facilitating transferring first layer 120 to a target site. For example, second layer 130 can include a polymer (e.g., a poly(ethylene glycol)), a biological material (e.g., phospholipids), or a metal (e.g., gold).

In general, second layer 130 can have any suitable thickness. For example, second layer 130 can have a thickness of at least about 0.1 μm (e.g., at least about 0.5 μm, at least about 1 μm, at least about 5 μm, or at least about 10 μm) or at most about 2,000 μm (e.g., at most about 1,500 μm, at most about 1,000 μm, at most about 500 μm, or at most about 100 μm).

Although FIG. 1 illustrates balloon 110 having a plurality of particles in first layer 120, in some embodiments, balloon 100 can include a plurality of fibers in lieu of or in addition to a plurality of particles in first layer 120. In general, the fibers in layer 120 can have the same configuration as the particles described above (e.g., a core-shell configuration) or include the same polymeric carrier and therapeutic agents as those in the particles described above. For example, when the particles in first layer 120 are replaced by a plurality of fibers, the circular cross-sections shown in FIG. 1 would represent the cross-section of the fibers. In such embodiments, therapeutic agent 126 in the fibers (e.g., in its crystalline form) can be in the shape of a rod. The average length of such rods in first layer 120 can range from at least about 0.1 μm (e.g., at least about 0.5 μm, at least about 1 μm, at least about 5 μm, or at least about 10 μm) or at most about 10 mm (e.g., at most about 5 mm, at most about 1 mm, at most about 0.5 mm, or at most about 0.1 mm).

The length of the fibers in first layer 120 can vary as desired. For example, the fibers can have an average length of at least about 0.1 μm (e.g., at least about 0.5 μm, at least about 1 μm, at least about 5 μm, or at least about 10 μm) or at most about 10 mm (e.g., at most about 5 mm, at most about 1 mm, at most about 0.5 mm, or at most about 0.1 mm).

Without wishing to be bound by theory, it is believed that an advantage of including a plurality of fibers in first layer 120 is that only a few places of a fiber need to adhere or bind to a target site (e.g., a tissue on a blood vessel wall) to keep the fiber attached to the target site and to make the therapeutic agent in the entire fiber (include the agent not located at the attaching point) available for uptake, thereby increasing the amount of the therapeutic agent that can be transferred and absorbed by the body.

In general, balloon wall 110 can be formed of a composite material that includes a polymeric material and optionally a filler. The optional filler can be uniformly dispersed within the polymeric material. The balloon can be formed using a dispersive polymer with good dispersion properties in combination with a balloon polymer that has properties particularly advantageous to balloons. The dispersive polymer can be a nucleophilic polymer, such as a polymer having amino groups (e.g., primary amino groups, secondary amino groups, or tertiary amino groups), hydroxyl groups and/or thiol groups. Examples include biologically-derived polymers such as chitosan and DNA. The balloon polymer can be, e.g., an electrophilic polymer, such as one having electrophilic groups (e.g., carboxylic acid groups) that react (e.g., ionically or covalently) with the dispersive polymer. Examples include polyacrylic acid and polyethylene terephthalates (e.g., a carboxylic acid functionalized polyethylene terephthalate). The polymers can be applied and combined on a pre-form substrate.

The optional filler can be an allotrope of carbon (e.g., diamond, graphite, C60, C70, C540, a single or multi-wall carbon tube, or amorphous carbon), a functionalized allotrope of carbon (e.g., functionalized with hydrogen bonding groups such as hydrogen bond acceptors and/or donors), a metal, a metal oxide (e.g., titanium dioxide), a metalloid oxide (e.g., silicon dioxide), a clay (e.g., kaolin), a ceramic (e.g., silicon carbide or titanium nitride), a polymeric material, different from the first or second polymeric material or a reaction product of the first and second polymeric materials, or mixtures or any of these fillers. If desired, the carbon nanotubes can encapsulate atoms other than carbon, such as a metal, which can, e.g., enhance radiopacity.

Other embodiments of balloon wall 100 have been described in, for example, commonly-owned co-pending U.S. Application Publication No. 2008-0287984.

In general, balloon 100 can be made by methods known in the art. For example, to prepare balloon 100, a bioadhesive polymer (e.g., polyhydroxybutyrate) can first be dissolved in an organic solvent (e.g., a water immiscible, volatile organic solvent such as dichloromethane) to form a solution. A therapeutic agent (e.g., paclitaxel) in a crystalline form (e.g., having an average particle size of 20 microns) can then be added to the solution to form a solution or a dispersion. The solution or dispersion can subsequently be emulsified by stirring (e.g., at an elevated temperature) and/or including an emulsifier (e.g., poly(vinyl alcohol)). After an emulsion is formed, the solvent can then be removed (e.g., by evaporation or extraction) to produce particles having a core-shell structure in which the core containing a therapeutic agent is encapsulated by a shell containing the bioadhesive polymer. The particles can optionally be washed, filtered, and dried. They can also be further treated by known methods so as to be free flowing. If desired, the particles can be milled to a suitable smaller particles size (e.g., 1,000 nm or below) by a known method (e.g., by ball milling using a nano mill ZETA RS available from Netzsch).

The particles thus formed can be applied on outer surface 111 of balloon 100 by methods known in the art. For example, the particles can be dispersed in a non-solvent (e.g., water or an aqueous solution) and then sprayed onto outer surface 111. As another example, the particles can be applied onto outer surface 111 by dipping balloon 110 in a dispersion containing the particles dispersed in a non-solvent. As another example, after outer surface 111 is pretreated with a sticky sugar solution, balloon 110 can roll over a tray of the particles, which would stick to outer surface 111.

When the particles have a core-shell structure in which an inert core (e.g., an inert core made of sugar particles) is surrounded by a shell containing a therapeutic agent dispersed in a polymeric carrier, the particles can be prepared by coating the inert core with a mixture of the therapeutic agent and polymeric carrier using a known technique, such as spaying coating or fluid bed coating (e.g., those used by GEA Niro, Soeborg, Denmark).

In some embodiments, particles (e.g., crystalline particles) of a therapeutic agent can first be dispersed in a solvent (e.g., water or an aqueous solution) containing a polymeric carrier. The dispersion thus obtained can then be sprayed onto outer surface 111 of balloon 110 to form a layer in which the therapeutic agent is dispersed (e.g., uniformly dispersed) in the polymeric carrier without forming any particle containing a core-shell structure.

When balloon 100 includes second layer 120, second layer 120 can be applied by coating outer surface 111 with a suitable polymer solution (e.g., a solution containing poly(ethylene glycol)) before first layer 110 is applied onto balloon. The coating can be performed by a liquid-based coating process, such as solution coating, ink jet printing, dip coating, spray coating, or roller coating.

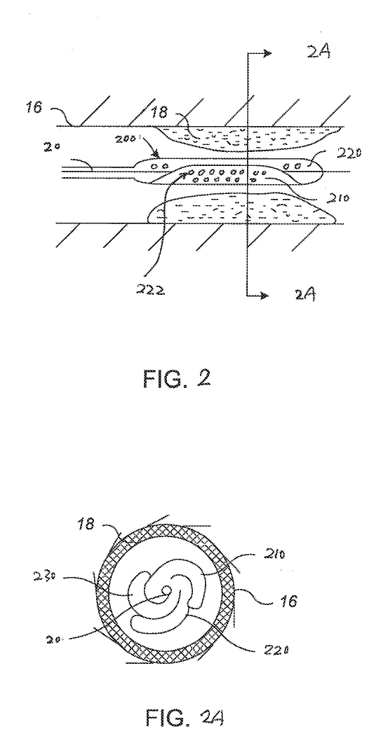

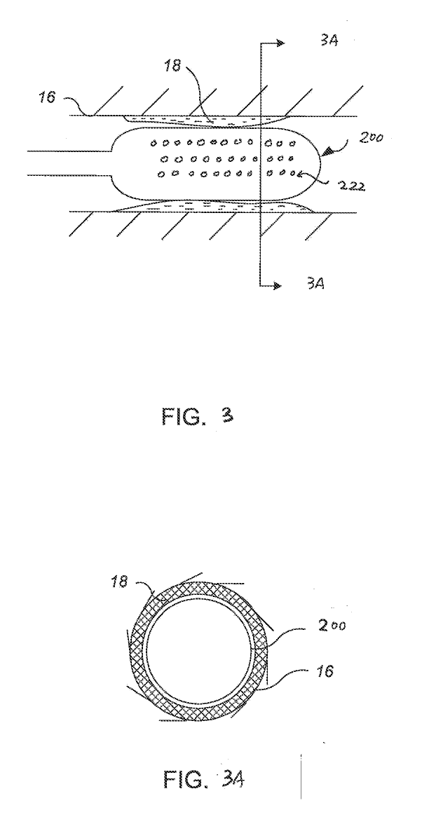

FIG. 2 illustrates a cross-sectional view of an exemplary balloon in a folded state within an occluded vessel and FIG. 2A illustrates an end view of the balloon in FIG. 2 in the vessel. Referring to FIGS. 2 and 2A, a balloon catheter 20 carrying a balloon 200 coated with the particles 222 described above is directed through a lumen 16 (e.g., a blood vessel such as the coronary artery) of a body over a guidewire (not shown) until balloon 200 reaches a target site, i.e., the region of an occlusion 18. To reduce the cross-sectional profile, balloon 200 is arranged into a series of lobes or wings 210, 220, and 230 which are wrapped about catheter 20. Referring to FIGS. 3 and 3A, when balloon 200 reaches the target site, it can be radially expanded by inflating with an inflation fluid (e.g., a gas or a liquid). Inflating balloon 200 causes its walls to press against the vessel wall of lumen 16 with the result that occlusion 18 is compressed and the vessel wall surrounding it undergoes a radial expansion. Upon contacting the vessel wall, the bioadhesive polymer in the particles 222 coated on the outer surface of the balloon adheres or binds to a tissue on the blood vessel wall.

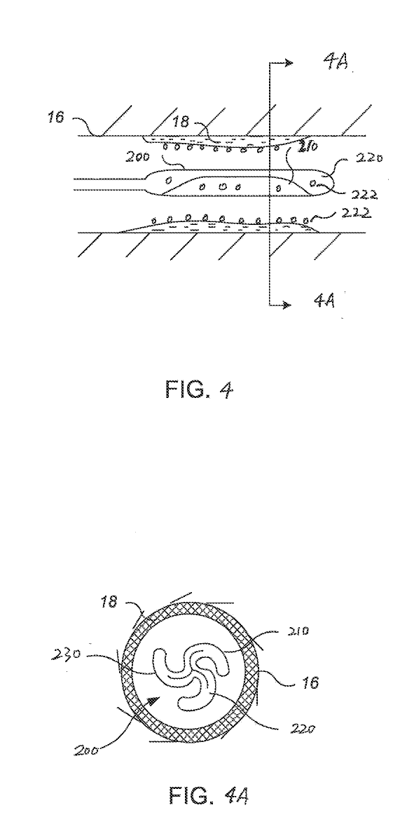

Referring to FIGS. 4 and 4A, after delivery of the therapeutic agent, the pressure is released from balloon 200. Balloon 200 then collapses into three lobes that curl over one another to configure balloon 200 into a compact shape, which can easily be removed from lumen 16. After balloon 200 is removed from lumen 16, particles 222 can still be attached to the tissues on the blood vessel wall due to the presence of the bioadhesive polymer. The bioadhesive polymer holds the therapeutic agent in particles 222 against the blood vessel walls until the therapeutic agent is taken up by the tissues (e.g., by diffusion through the bioadhesive polymer). After the therapeutic agent is taken up by the body, the bioadhesive polymer can be degraded into harmless small molecules, which can either be absorbed by, or discharged from, the body.

### Example 1

A coating composition is prepared by dissolving or dispersing 5-30 wt % of a bioadhesive polymer (i.e., polyhydroxybutyrate) in 20-90 wt % of a gel composition (containing 2 wt % of poly(ethylene oxide), 0.2 wt % neopentyl glycol, 19.6 wt % water, and 78 wt % isopropanol). Paclitaxel nanoparticles are then added into the coating composition such that the drug-bioadhesive polymer ratio reaches 5-50 wt %.

A balloon is inflated to a certain pressure and then unfolded to form a cylindrical shape. The inflated balloon is subsequently dipped into the coating composition obtained above and withdrawn slowly so that an even coating is applied onto the balloon. The isopropanol is allowed to be evaporated to form a coating containing paclitaxel nanoparticles dispersed in the bioadhesive polymer. The thickness of the coating is approximately 1 mm. The balloon is then deflated, refolded, packaged, and sterilized. The amount of paclitaxel deposited on the balloon is obtained from the mass of the coating, which is determined by weighing the balloon before and after the coating is applied.

The coated balloon is inserted into the artery of a patient using a known minimally invasive surgical techniques, that is, it is advanced along a guidewire to the arterial lesion to be treated. The balloon is expanded when in position at the lesion so that the balloon surface expands against the lesion. The pressure applied by the balloon causes the coating to transform into a low viscosity material spreading against the artery wall and lesion. The bioadhesive polymer in the coating then helps the paclitaxel particles adhere to the artery wall. The balloon is deflated and withdrawn from the body, leaving a coating layer containing the paclitaxel, bioadhesive polymer, and gel on the artery wall at the lesion site. Upon adhering to the artery wall, paclitaxel starts to diffuse into the artery wall to prevent restenosis.

### Example 2

A dispersion of drug nanoparticles is prepared (as in Example 1 by ball milling) to a known size distribution (e.g. 50-150 nm) and measured using the Malvern Mastersizer.

The bioadhesive polymer PolyLacticAcid is dissolved in a low boiling point (acetone) solvent to make a near-saturated solution. A special spray nozzle with a dual concentric orifice is used to spray both liquids at the same time—the drug suspension flows through the central orifice and the biopolymer solution flows through the outer annular orifice. A nozzle like this is the Sono-Tek ultrasonic dual liquid feed nozzle (www.sono-tek.com/nanotechnology/page/9/1). When both liquids are sprayed using this nozzle the solvent evaporates quickly and nanoparticles are formed with a drug core and a biopolymer shell. The core/shell particles can be collected after spraying into a container and then added to the gel material (in a 1:1 w/w ratio) before applying the whole gel/particle mixture to the balloon using dipping. Alternatively the gel is applied first to the balloon and the drug core/shell particles are directly sprayed onto the gel surface using the dual liquid feed nozzle.

Other embodiments are in the claims.

What is claimed is:

1. A medical device, comprising:
a drug-delivery balloon comprising a balloon wall having an outer surface and a first layer supported by the outer surface;
wherein the first layer comprises a plurality of particles, each particle comprises a therapeutic agent and a polymeric carrier, and the polymeric carrier comprises a polymer capable of adhering or binding to a tissue on a wall of a blood vessel.

2. The device of claim 1, wherein each particle comprises a core encapsulated by a shell, the core comprises the therapeutic agent, and the shell comprises the polymeric carrier.

3. The device of claim 1, wherein the therapeutic agent is dispersed in the polymeric carrier in each particle.

4. The device of claim 1, wherein the plurality of particles have an average diameter of from about 20 nm to about 1,000 nm.

5. The device of claim 1, wherein the polymer is a biodegradable polymer.

6. The device of claim 1, wherein the polymeric carrier comprises a polyhydroxyalkanoate, a polylactone, a polylactic acid, polyglycolic acid, a cyanoacrylate-based polymer, a polyacrylate, a poly(vinyl alcohol), a poly(ethylene glycol), or a copolymer or mixture thereof.

7. The device of claim 1, wherein the polymeric carrier comprises a polyhydroxybutyrate, a polylactic acid, a poly(methyl methacrylate), a poly(vinyl alcohol), a poly(ethylene glycol), or a copolymer or mixture thereof.

8. The device of claim 1, wherein the polymer has a number average molecular weight of from about 10,000 g/mol to about 75,000 g/mol.

9. The device of claim 1, wherein the polymer has a shear viscosity of from about 5,000 centipoises to about 2×10⁶ centipoises.

10. The device of claim 1, wherein the first layer has an elastic modulus of from about 10 kPa to about 10 MPa.

11. The device of claim 1, wherein the polymer carrier is capable of maintaining at least about 25 wt % of the therapeutic agent at a target site in a blood vessel for at least about 14 days after the balloon is withdrawn from the blood vessel.

12. The device of claim 1, wherein the therapeutic agent is therapeutically effective in inhibiting restenosis.

13. The device of claim 1, wherein the therapeutic agent comprises paclitaxel, everolimus, or a derivative thereof.

14. The device of claim 1, wherein the therapeutic agent is in a crystalline form.

15. The device of claim 1, wherein each particle comprises from about 5 wt % to about 90 wt % of the therapeutic agent.

16. The device of claim 1, wherein the first layer is a discontinuous layer.

17. The device of claim 1, wherein the balloon further comprises a second layer between the first layer and the outer surface of the balloon wall, the second layer being capable of inhibiting binding of the first layer to the outer surface of the balloon wall.

18. The device of claim 17, wherein the second layer comprises a poly(ethylene glycol), a phospholipid, or a metal.

19. The device of claim 1, wherein at least about 50 wt % of the therapeutic agent remains on the outer surface of the balloon wall when the balloon reaches a target site in a blood vessel.

20. The device of claim 1, wherein the balloon is capable of transferring at least about 20 wt % of the therapeutic agent to a target site in a blood vessel.

21. A medical device, comprising:
a drug-delivery balloon comprising a balloon wall having an outer surface and a first layer supported by the outer surface;
wherein the first layer comprises a plurality of fibers and each fiber comprises a therapeutic agent and a polymeric carrier.

22. A medical device, comprising:
a drug-delivery balloon comprising a balloon wall having an outer surface and a first layer supported by the outer surface;
wherein the first layer comprises a therapeutic agent and a polymeric carrier, and the polymeric carrier comprises a polymer capable of adhering or binding to a tissue on a wall of a blood vessel.

23. A medical device, comprising:
a drug-delivery balloon comprising a balloon wall having an outer surface and a first layer supported by the outer surface;
wherein the first layer comprises a therapeutic agent and a polymeric carrier, and the polymer carrier is capable of maintaining at least about 25 wt % of the therapeutic agent at a target site in a blood vessel for at least about 14 days after the balloon is withdrawn from the blood vessel.
